(19) United States
(12) Patent Application Publication
(10) Pub. No.: US 2013/0272963 A1
(43) Pub. Date: Oct. 17, 2013

(54) Cholesterol Efflux Assay Probe Formulations, Methods of Making and Using

(71) Applicant: THE OHIO STATE UNIVERSITY, Columbus, OH (US)

(72) Inventors: Sanjay Rajagopalan, Columbus, OH (US); Markus A. Badgeley, Columbus, OH (US); Andrei Maiseyeu, Columbus, OH (US)

(73) Assignee: The Ohio State University, Columbus, OH (US)

(21) Appl. No.: 13/861,832

(22) Filed: Apr. 12, 2013

Related U.S. Application Data
(60) Provisional application No. 61/623,307, filed on Apr. 12, 2012.

(51) Int. Cl. C12Q 1/60; A61K 31/138; A61K 31/4439; A61K 31/337; A61K 9/14; A61K 49/00

(52) U.S. Cl. CPC C12Q 1/60; A61K 9/14; A61K 49/00; A61K 31/4439; A61K 31/337; A61K 31/138
USPC 424/9.1; 435/11; 424/498; 514/342; 514/449; 514/648; 514/1.1; 435/287.1

(57) ABSTRACT

A cholesterol efflux assay probe formulation having a core comprised of a biocompatible hydrophobic material at least partially coated with a sphingomyelin/cholesterol layer, methods of making and methods of using are described.

### RELATED APPLICATIONS

This application claims priority to U.S. Provisional Patent Application Ser. No. 61/623,307 filed Apr. 12, 2012, the disclosure of which is incorporated herein by reference, in its entirety.

### GOVERNMENT SUPPORT

The invention was made with no government support and the government has no rights in the invention.

### BACKGROUND

Atherosclerosis, the buildup of plaque in artery walls, is the leading cause of global morbidity and mortality. Therapies to induce “regression” of plaque build-up in arteries are thought to hold considerable promise. The assessment of regression of plaque requires insights into Reverse Cholesterol Transport (RCT), a key anti-atherosclerotic process. RCT involves the elimination of cholesterol deposits out of cells (called macrophages) in arteries and peripheral tissues to the feces through transport by high density lipoprotein (HDL). HDL transports cholesterol to the liver for excretion. In contrast, low density lipoprotein (LDL) transports cholesterol towards peripheral tissues. LDL is associated with disease within the arteries and is thus known as “bad cholesterol.”

Direct in-vivo assessment of cholesterol trafficking from cells in the blood vessels may be laborious, expensive, not readily standardizable, not easy to perform in humans, and may not provide readily interpretable results. As such, in-vitro assessment of the effectiveness of cholesterol acceptors such as HDL to remove cholesterol from cholesterol loaded cells may be performed as a surrogate to investigate RCT. In the past, such in-vitro assessment provided a read out of RCT using in-vitro loading of macrophages with radiolabeled cholesterol in order to investigate HDL dysfunction in a variety of diseases and pathological conditions. However, the current RCT assay methods are variable, time consuming, and involve radio-active methods. Thus there is a need for robust, reproducible, cost-efficient, and high-throughput methods that allow for rapid assessment of RCT.

There is a further need for a method that is simple, yet allows for conditions mimicking the native transport mechanisms. There is a particular need for this since the synthesis of acetylated LDL (acLDL) and other probes now used in RCT assays is lengthy, laborious, and often involves experiments with human blood and radioactivity which may pose significant hazards. Prior Art FIG. 2 illustrates one workflow chart for the synthesis of acLDL probes, where there are more than 7 synthetic steps (with purification) needed in order to obtain acLDL suitable for RCT assays. This synthetic method is time-consuming, costly, and hazardous. Further, lipid composition of acLDL cannot be changed; only limited modifications are allowed in order to keep lipoprotein structure intact. LDL and acLDL are commercially available, but very expensive and are only made by biotech companies “upon request” due to lipoprotein short shelf-life.

### SUMMARY

In a first aspect, there is provided herein a hydrophobic molecule efflux assay probe formulation, comprising at least one nanoparticle having a core comprised of a biocompatible hydrophobic material at least partially coated with at least one type of hydrophobic molecule, such as lipids. In certain embodiments, the nanoparticles are coated with sphingomyelin and cholesterol. In certain embodiments, the core is polystyrene or another material capable of incorporating cholesterol molecules. In certain embodiments, the core has a diameter in the range of about 25 nm to about 50 nm, and in certain embodiments the core has a mean diameter size of about 31 nm. In certain embodiments, the core is at least partially porous to allow for passive adsorption of lipids and hydrophobic molecules on the surface of the formulation.

In certain embodiments, the formulation further includes at least one of: a therapeutic agent, a diagnostic agent, or a contrast agent, at least partially encapsulated in the formulation.

In certain embodiments, the formulation has a Zeta potential in the range of from about −10 mV to about −100 mV. In certain embodiments, the formulation is stable at cell culture conditions in the presence of high salt or high protein content.

In certain embodiments, the formulation is loaded with a drug selected from cholesterol drugs, anticancer agents, antibacterial agents, antiviral agents, autoimmune agents, anti-inflammatory agents, cardiovascular agents, antioxidants, and therapeutic peptides. In certain embodiments, the drug is Rosiglitazone, Paclitaxel, or Tamoxifen.

In certain embodiments, the cholesterol in the formulation is labeled with at least one of a radioactive label, a fluorescent label, or an isotopic label. The core can also comprise a fluorescent substance.

In another aspect, there is provided herein a method for making an efflux assay probe formulation, comprising: forming a mixture of hydrophobic molecules; adding the mixture to an aqueous solution of 20-25 nm polystyrene latex nanoparticles to form a suspension; and sonicating the suspension to yield the formulation described herein. In certain embodiments, the mixture of hydrophobic molecules comprises sphingomyelin and cholesterol. In certain embodiments, the method involves labeling cholesterol with a radioactive label, a fluorescent label, or an isotopical label. In certain embodiments, the method involves incorporating at least one of: a therapeutic agent, a diagnostic agent, or a contrast agent into the formulation, wherein the at least one of the therapeutic agent, the diagnostic agent, and the contrast agent are at least partially encapsulated in the formulation.

In another aspect, there is provided herein a method for conducting a reverse cholesterol transport assay, comprising loading cells with an efflux assay probe formulation, treating the cells with a cholesterol acceptor such as HDL, and then analyzing the cholesterol transported by the acceptor into the extracellular media. The cholesterol can be labeled, and the method can further involve conducting real-time monitoring of cholesterol efflux by monitoring Förster Resonance Energy Transfer (FRET) effects.

Further disclosed herein is a method of delivering a drug to a subject in need thereof. The method involves incorporating an effective amount of a drug into an efflux assay probe formulation, then administering the drug to the subject. The drug can be any cholesterol drug, anticancer agent, antibacterial agent, antiviral agent, autoimmune agent, anti-inflammatory agent, cardiovascular agent, antioxidant, or therapeutic peptide.

Further disclosed is a method of delivering a therapeutic or diagnostic agent to a target site, involving incorporating a therapeutic or diagnostic agent into an efflux assay probe formulation, then delivering the formulation to a target site, wherein the delivery includes one of (i) engulfment of the formulation within the target site, (ii) release of a diagnostic agent incorporated within the formulation to the target site, or (iii) release of a therapeutic agent incorporated within the formulation to the target site. In certain embodiments, the target site is atherosclerotic plaque tissue. The method can further include imaging the target site.

Further provided herein is a method of monitoring cholesterol efflux in real time. The method involves loading cells with nanoparticles having a fluorescent core and fluorescently labeled cholesterol, then monitoring the FRET efficiency of the nanoparticles in order to monitor cholesterol efflux in real time. In certain embodiments, the fluorescent core is red fluorescent polystyrene. In certain embodiments, the cholesterol is labeled with boron-dipyrromethene (BODIPY). In certain embodiments, the nanoparticles further comprise sphingomyelin. In certain embodiments, the method further comprises the step of incubating the cells with a cholesterol acceptor.

In another aspect, there is provided herein a ready-to-use RCT assay. In some embodiments, the assay comprises a supply of cells pre-loaded with a CHEAP formulation, and one or more cholesterol acceptors. The assay can be one or more of: fluorescence-, radioisotope- and/or mass-isotope-based assays. In certain embodiments, the one or more cholesterol acceptors includes HDL. In other embodiments, the assay comprises a multiwell plate of cells separate and a CHEAP formulation in separate containers. In other embodiments, the assay comprises a supply of cells either preloaded with, or housed separately from, an efflux assay probe formulation comprising lipids other than cholesterol.

In another aspect, the efflux assay probe formulations provided herein can be used to determine a lipid efflux profile by incorporating a variety of lipids into the formulation used for an assay. In some embodiments, assessing the lipid efflux profile of a subject includes analyzing total cholesterol, cholesterol ester, HDL, LDL, intermediate density lipoprotein (IDL), very low density lipoprotein (VLDL), triglycerides, phospholipids selected from the group consisting of sphingolipids and phosphatidyl choline, and combinations thereof. In some embodiments, the sphingolipids are selected from the group consisting of sphingosines, ceramides, and sphingomyelins. Thus, in some embodiments, assessing the lipid efflux profile includes analyzing cholesterol ester, sphingosines, ceramides, sphingomyelins, and phosphatidyl choline. It certain embodiments, a lipid efflux profile includes analyzing only cholesterol. The lipid efflux profile can be obtained from macrophage cell types, macrophage-like cells, intact artery tissues, or in-vitro mobilization to plasma. Lipid efflux profiles can be used in methods to diagnose and/or identify subjects with a reverse cholesterol transport deficiency and distinguish responders from non-responders to a treatment for an RCT deficiency-related condition.

Further provided are methods of prognosing, diagnosing, and/or predicting a response to treatment of a condition associated with a deficiency in a reverse cholesterol transport pathway in a subject comprising the steps of (i) providing a population of cells from the subject, (ii) loading the cells with an efflux probe formulation described herein, (iii) assessing the lipid efflux profile, (iv) determining whether there is a deficiency in the reverse cholesterol transport pathway of the subject, and (v) prognosing, diagnosing, and/or predicting a response to treatment of the condition associated with a deficiency in a RCT pathway, wherein the prognosing, diagnosing, and/or predicting is based on the determining in step (iv).

Further provided are methods of screening compounds for treatment of a condition associated with reverse cholesterol transport deficiency and/or assessing the risk of toxicity of a treatment of a condition associated with a reverse cholesterol transport deficiency. In some embodiments, the methods involve the steps of (i) providing cells from a subject, (ii) contacting the cells with one or more compounds that are possible candidates for the treatment of a condition associated with reverse cholesterol transport deficiency and/or where the one or more compounds are used in the treatment of a condition associated with reverse cholesterol transport deficiency, (iii) assessing the lipid efflux profile in the cells treated with the compound or a medium comprising the cells, using an efflux probe formulation described herein, and (iv) selecting the one or more compounds for treatment of the condition associated with reverse cholesterol transport deficiency and/or determining the toxicity of a treatment of the condition associated with reverse cholesterol transport deficiency, where the selecting and/or the determining are based on the assessing from step (iii). Suitable media for comprising the cells include, but are not limited to, tissue, organs, blood, serum, plasma, body fluid, or culture media.

Further provided are methods of prognosing, diagnosing, and/or predicting a response to a treatment of a condition associated with a deficiency in a reverse cholesterol transport pathway in a subject. In some embodiments, the methods comprise (i) administering to a subject an efflux probe formulation described herein, (ii) assessing the lipid efflux profile in at least one cell from the subject, (iii) determining whether there is a deficiency in the reverse cholesterol transport pathway of the subject, where the determining is based on the assessing of the lipid efflux profile, and (iv) prognosing, diagnosing, and/or predicting a response to a treatment of the condition associated with a deficiency in a reverse cholesterol transport pathway, where the prognosing, diagnosing, and/or predicting is based on the determining in step (iii).

Further disclosed is a method of diagnosing a subject with a deficiency in the RCT pathway. In some embodiments, the method involves the steps of (i) isolating cells from a subject, (ii) contacting the cells with a compound that specifically modulates a reverse cholesterol transporter pathway, (iii) assessing the lipid efflux profile of the cells treated with the compound as compared to the lipid efflux profile of a control cell of the same type, using an efflux assay probe formulation described herein, and (iv) diagnosing a condition associated with a deficiency in a reverse cholesterol transport pathway, where the diagnosing is based on the assessing in step (iii).

In some embodiments, the methods described herein may further comprise the step of comparing a lipid efflux profile to a predetermined threshold value. The methods may also further comprise treating a RCT-related disease by administering to the subject in need thereof an effective amount of a therapeutic agent, such as a modulator that is specific for a reverse cholesterol transporter.

Further disclosed is a kit for the preparation of a CHEAP formulation. In certain embodiments, the kit has a first container with a mixture of sphingomyelin and cholesterol, a second container with a biocompatible hydrophobic material, and, optionally, a sonicator. In certain embodiments, the biocompatible hydrophobic material is polystyrene. In certain embodiments, the kit further includes a therapeutic agent, a diagnostic agent, or a contrast agent.

Additional advantages, objects, and features of the invention will be set forth in part in the description which follows and in part will become apparent to those having ordinary skill in the art upon examination of the following, or may be learned from practice of the invention. The objects and advantages of the invention may be realized and attained as particularly pointed out in the appended claims.

### BRIEF DESCRIPTION OF THE FIGURES

The patent or application file may contain one or more drawings executed in color and/or one or more photographs. Copies of this patent or patent application publication with color drawing(s) and/or photograph(s) will be provided by the U.S. Patent and Trademark Office upon request and payment of the necessary fees.

FIG. 1: Schematic illustration of in vitro Reverse Cholesterol Transport (RCT) process: Step 1) Cells are loaded with cholesterol-containing particles; Step 2) Particles are degraded in lysosomes due to action of phospholipases and low pH, releasing free cholesterol; Step 3) Free cholesterol is transported to the cell membrane; Step 4) Cholesterol acceptors transfer cholesterol outside of the cell; and, Step 5) Extracellular media is subjected to cholesterol analysis after extraction of free cholesterol.

Prior Art FIG. 2: Schematic illustration of a prior art multistep synthesis of a acLDL probe is lengthy and involves multiple purification stages. The synthesis is started from whole blood or human plasma which is centrifuged to separate out the plasma. While commercial sources of LDL and acLDL are available, high cost and “only upon request” purchasing prevents most research groups from using them on a widespread basis.

FIG. 3A: Schematic illustration of a comparison of a acLDL probe (left) and a cholesterol efflux assay probe (CHEAP) (right) formulation. The probes generally range in size from about 20 nm to about 30 nm, and are capable of cholesterol loading. While the acLDL probe core consists of lipids and apoB protein, the CHEAP probe is formed of at least one biocompatible material (such as polystyrene) and at least one lipid (such as sphingomyelin) mixed with cholesterol.

FIG. 3B: Schematic illustration of synthesis of a CHEAP formulation. Particles are made by self assembly in water solutions upon sonication of a lipid-cholesterol mixture and hydrophilic polystyrene nanoparticles. In certain embodiments, no further purification steps are needed and the CHEAP composition is useful in RCT assays immediately after synthesis.

FIG. 4: Bubble chart illustrating a screening strategy for preparation of one embodiment of a CHEAP formulation. Screening of 25 formulations (e.g., sphingomyelin vs. cholesterol content) was performed with respect to multiple physicochemical properties such as: size (represented by bubble size), Förster Resonance Energy Transfer (FRET) efficiency (multicolor heat-map), and zeta potential/polydispersity ratio (negative number over selected formulations). Results identified one optimized particle with mean size similar to native LDL (32.3 nm), high FRET efficiency and superior colloidal stability.

FIG. 5: Graph illustrating that CHEAP formulations containing cholesterol are stable at cell culture conditions and do not release a substantial amount of cholesterol while outside the cell. A tritium-labeled CHEAP formulation was incubated in DMEM at 37° C., and cholesterol release was monitored over time. A very small amount of radioactivity (˜1% of total) was found in exterior DMEM, as was shown by dynamic dialysis experiments.

FIGS. 6A-6B: CHEAP formulations can be loaded with a variety of therapeutic drugs:

FIG. 6A: Graphs showing three biomedical research hydrophobic drugs tested with respect of CHEAP-drug loading capacity. RSG=Rosiglitazone. PAX=Paclitaxel. TAM=Tamoxifen.

FIG. 6B: Graph showing the release of RSG from a RSG-loaded CHEAP formulation in cell-culture media at 37° C. RSG release was analyzed via LC-MS.

FIG. 7: Graph showing fluorescence emission spectra profiles of a BODIPY-cholesterol loaded (Ex 465 nm; Em 520 nm), nile-red core derivatized (Ex 570 nm; Em 605 nm) CHEAP formulation at different excitation wavelengths. CHEAP emission at 605 nm upon excitation at 465 nm shows efficient Förster Resonance Energy Transfer (FRET) between the cholesterol and the core.

FIGS. 8A-8B: Macrophages were treated with a fluorescent acLDL or a CHEAP formulation, and subsequently stained for endosomes (FIG. 8A) or for lysosomes (FIG. 8B). The CHEAP formulations appear red, nuclei appear blue, and the endosomes or lysosomes appear green.

FIG. 9: Graph showing a comparison of acLDL vs. CHEAP cholesterol loading in radio-RCT assays. The effect of HDL concentration on cholesterol efflux was studied with two probes. Cells were loaded with radioactively labeled cholesterol by incubation with acLDL (blue series) or CHEAP (red series) for 4 h. After cholesterol loading, cells were treated with a concentration gradient of HDL (a cholesterol acceptor) for 4 h. Percent (%) efflux was defined as the amount of cholesterol in the media divided by the total amount of cholesterol in the cells.

### DETAILED DESCRIPTION OF THE INVENTION

For convenience, certain terms employed in the specification, examples, and appended claims are collected here, before further description of the invention. These definitions should be read in light of the remainder of the disclosure and understood as by a person of skill in the art. Unless defined otherwise, all technical and scientific terms used herein have the same meaning as commonly understood by a person of ordinary skill in the art.

The articles “a” and “an” are used to refer to one or to more than one (i.e., to at least one) of the grammatical object of the article. By way of example, “an element” means one element or more than one element.

The term “plurality” means more than one.

The terms “comprise” and “comprising” are used in the inclusive, open sense, meaning that additional elements may be included.

The term “including” is used to mean “including but not limited to”. “Including” and “including but not limited to” are used interchangeably.

A “diagnostic” or “diagnostic agent” is any chemical moiety that may be used for diagnosis.

For example, diagnostic agents include imaging agents containing radioisotopes such as indium or technetium, contrasting agents containing iodine or gadoliniums, or the like.

“Diagnosis” is intended to encompass diagnostic, prognostic, and screening methods.

A “patient,” “subject” or “host” to be treated by the subject method may mean either a human or non-human animal. The methods described herein are generally carried out on mammalian subjects, such as humans. The term “mammal” is known in the art, and exemplary mammals include humans, primates, bovines, porcines, canines, felines, rodents (e.g., mice and rats), farm animals, rabbits, and sport animals.

The term “latex” as used herein refers to an emulsion or stable dispersion of microparticles in an aqueous medium.

The term “phospholipid” as used herein refers to any lipid containing a phosphate group. Examples of phospholipids include, but are not limited to, phosphatidylcholine, phosphadtidylethanolamine, phosphatidic acid, phosphatidylinositol, phosphatidylglycerol, cariolipin, sphingomyelin, and phosphatidylserine.

The term “incorporation” as used herein refers to imbibing or adsorbing a molecule, such as a diagnostic agent or a therapeutic agent, onto a nanoparticle. The term “encapsulation” as used herein refers to the enclosure of a molecule, such as a diagnostic agent or therapeutic agent, inside a nanoparticle. Encapsulation may occur, in one example, by synthesis of nanoparticles in the presence of a liquid solution containing the molecule(s) to be encapsulated.

The term “stable” as used herein refers to compounds which possess stability sufficient to allow manufacture and which maintain the integrity of the compound for a sufficient period of time to be detected and preferably for a sufficient period of time to be useful for the purposes detailed herein.

The present invention will now be described with occasional reference to the specific embodiments of the invention. This invention may, however, be embodied in different forms and should not be construed as limited to the embodiments set forth herein. Rather, these embodiments are provided so that this disclosure will be thorough and complete, and will fully convey the scope of the invention to those skilled in the art. All publications, patent applications (and any patents that issue thereon, as well as any corresponding published foreign patent applications), patents, and other references mentioned herein are incorporated by reference in their entirety. It is expressly not admitted, however, that any of the documents incorporated by reference herein teach or disclose the present invention.

Efflux Assay Probe Formulations

Described herein are efflux assay probe formulations having synthetic nanoparticles capable of delivering labeled cholesterol intracellularly. In a particular aspect, the described assay probe formulations are generally referred to herein as CHolesterol Efflux Assay Probe (CHEAP) formulations, and are useful in cholesterol transport investigations such as RCT assays. It is to be understood that the term “CHEAP” is used herein for illustrative purposes only, and that uses of hydrophobic molecules other than or in addition to cholesterol are within the contemplated scope of the present invention. The formulations are discussed with reference to CHEAP formulations for ease of explanation only.

A synthetic nanoparticle formulation for RCT assays should meet the following criteria: structural similarity of the nanoparticles; high cholesterol loading capacity; fast and efficient engulfment of the nanoparticles by cells; high-efficiency scavenger receptor and/or LDL-receptor-mediated uptake; entrapment of the nanoparticles in lysosomes; fast degradation of the nanoparticles in lysosomes followed by cholesterol release into the cell cholesterol pool; and biocompatibility. Consistent with this, the CHEAP formulations generally comprise nanoparticles having a biocompatible hydrophobic core at least partially coated with a lipid-cholesterol mixture. The core of the nanoparticles is a robust, stable biocompatible material that is hydrophobic and somewhat porous in nature. This allows for the passive adsorption of lipids and hydrophobic molecules on the surface of the nanoparticles, allowing the nanoparticles to be coated with a lipid-cholesterol layer. In certain embodiments, the core is polystyrene. In certain embodiments, the lipid-cholesterol mixture comprises a phospholipid and cholesterol. In certain embodiments, the phospholipid is sphingomyelin because of its high affinity to bind cholesterol. Many formulations of these nanoparticles are possible by varying the makeup of the phospholipid and cholesterol components in the lipid-cholesterol mixture. In one non-limiting example, the formulation has a concentration of sphingomyelin of about 1.25 mg/mL, and a concentration of cholesterol of about 0.25 mg/mL. Additional lipids such as, but not limited to, cholesterol esters, triglycerides, sphingolipids, or combinations thereof can also be present in the lipid-cholesterol mixture or otherwise incorporated into the CHEAP formulation.

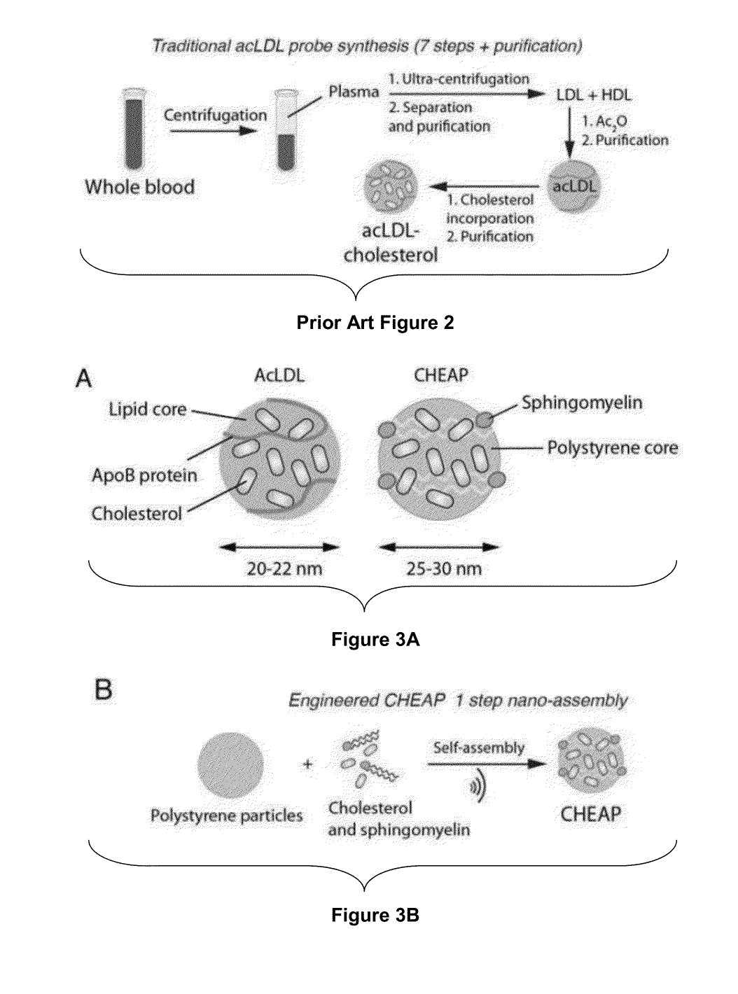

Synthesis of the CHEAP nanoparticles involves a facile, single self-assembly step. The route to CHEAP synthesis is shown in FIG. 3A. In one embodiment, a dried mixture of cholesterol (labeled or label-free) and sphingomyelin is treated with an aqueous solution of 20-25 nm polystyrene latex nanoparticles, which can be bulk-produced or obtained commercially, to form a suspension. The suspension is sonicated at high power, resulting in CHEAP nanoparticles that require no further processing or purification steps. The resulting CHEAP nanoparticles have a size ranging from about 20 nm to about 2500 nm. In certain embodiments, the nanoparticles have a size ranging from about 25 nm to about 50 nm. In one embodiment, the nanoparticles have a mean diameter size of about 31 nm. In certain embodiments, the CHEAP nanoparticles have a zeta potential/polydispersity ratio of about −137 and a FRET efficiency of about 0.75. The zeta potential/polydispersity ratio is an indicator of colloidal stability, with smaller ratios representing particles of highly negative charge and low polydispersity index (uniform particle size distribution). The CHEAP nanoparticles are stable at 37° C. cell culture conditions, as well as room temperature or +4° C., giving CHEAP formulations an advantageous shelf life.

The CHEAP formulations, having fully synthetic nanoparticles that are capable of delivering labeled cholesterol cargo into a cell, are versatile and have several advantages over known RCT assay probes. CHEAP formulations have dramatically improved product purity. CHEAP nanoparticle self-assembly allows for mass production with faster and inexpensive synthesis. CHEAP formulations enable real-time cholesterol release monitoring and allow for the ability to incorporate therapeutic and experimental drugs with simultaneous intracellular delivery. CHEAP-assisted RCT assays can be automated with the use of multiplate fluorescence readers, thereby enabling facile clinical sample testing. The CHEAP formulation is fully synthetic, meaning there are no health hazards associated with the preparation of CHEAP formulations. The CHEAP formulation can be finely tuned to incorporate a variety of fluorescent, radio, mass-isotope, paramagnetic, and other reporters of cholesterol trafficking, making CHEAP significantly versatile. The CHEAP formulation is stable in cell-culture conditions, as well as at room temperature, for many months with excellent shelf-life stability. Additionally, the end user of the CHEAP formulation does not need an institutional review board (IRB) or independent ethics committee (IEC) approval to purchase and use a CHEAP formulation, as opposed to purchasing human blood (acLDL synthesis).

In other embodiments, the efflux assay probe formulation comprises nanoparticles layered with lipids other than, or in addition to, cholesterol. Suitable lipids for use in these formulations include, but are not limited to, triglycerides, diglycerides, monoglycerides, fats, sterols, waxes, and phospholipids. In certain embodiments, these formulations are synthesized in the same manner as CHEAP formulations, with the desired lipids added in a mixture with a phospholipid, such as sphingomyelin, to polystyrene nanoparticles to form a suspension, the suspension then being sonicated to form efflux assay probe formulation nanoparticles ready for use in assays without further processing or purification.

Assays

CHEAP nanoparticles are taken up by cells in a manner similar to natural LDL. The nanoparticles accumulate in lysosomal compartments of the cells, and are degraded by the lysosomes to release cholesterol in the intra-cellular pool. Lysosomal degradation of the nanoparticles is caused by the action of phospholipases and the low pH environment of the lysosomes. Once released, the cholesterol is transported by shuttle proteins to the cell membrane, where the cholesterol is available for transfer to cholesterol acceptors such as HDL. This process enables in-vitro assessment of the RCT mechanism. Thus, provided herein are ready-to-use RCT assays. In one embodiment, an assay comprises CHEAP-preloaded murine cells for easy, one-step RCT investigations, and a cholesterol acceptor, such as HDL. The assays can comprise pre-made multiwell plates, and can be shipped in dry ice. To use such an assay, the user adds the cholesterol acceptor to the cells, incubates, and then performs the analysis.

In certain embodiments, the assays comprise multiwell plates of cells separate from a CHEAP formulation, such that the user adds an enclosed CHEAP formulation to the cells, then adds a cholesterol acceptor, incubates, and performs the analysis. Any of the assays described herein can be fluorescence-, radioisotope-, or mass-isotope-based assays for cholesterol efflux detection, depending on the labeling of the cholesterol therein.

In other embodiments, the assays comprise multiwell plates of cells loaded with, or separate from, an efflux assay probe formulation comprising nanoparticles layered with one or more lipids in place of, or in addition to, cholesterol. These assays can be used to analyze a lipid efflux profile. As with other embodiments, these assays also provide for easy, one-step investigations of lipid efflux. Additionally, the assays can comprise labeled lipids to enable fluorescence-, radioisotope-, or mass-isotope-based assays for lipid efflux detection.

CHEAP formulations are useful to assist and improve the workflow in RCT assays. The synthetic CHEAP formulation provides nanoparticles having generally uniform sizes; a high cholesterol loading capacity; fast and efficient engulfment by cells; high-efficiency scavenger receptor and/or LDL-receptor-mediated uptake; entrapment and fast degradation in lysosomes, followed by cholesterol release into the cell cholesterol pool; and biocompatibility. CHEAP assays therefore make cholesterol transport investigations faster and less laborious, less expensive, more consistent, and more controllable.

Real-Time Monitoring

In another particular aspect, there is described herein a method of using a CHEAP formulation in assisted endpoint and/or real-time cholesterol efflux monitoring; for example, by means of fluorescence detection, radio assay data collection, and Förster Resonance Energy Transfer (FRET). FRET efficiency is inversely proportional to the distance between two chromophores. In the context of CHEAP nanoparticles comprising a fluorescent core and fluorescently labeled cholesterol, a higher FRET efficiency indicates a close association of the lipid-cholesterol mixture on particle's surface to the core of the particles. The cholesterol can thus be tracked by monitoring the loss of FRET effects. Any CHEAP formulation can be synthesized with a fluorescent core and fluorescently labeled cholesterol to enable such monitoring. Examples of suitable fluorescent labels for cholesterol include, but are not limited to, fluorescent dyes such as BODIPY.

Further provided is an assay for the real-time monitoring of cholesterol efflux. The assay for real-time monitoring of cholesterol efflux comprises an assay as described above and further comprises a fluorescence reader for monitoring FRET efficiency in real-time.

Drug Loading

In another particular aspect, there is described herein a CHEAP formulation for the delivery of cholesterol drugs which target biochemical pathways implicated in cholesterol trafficking. Such formulations incorporate hydrophobic drug molecules rather than, or in addition to, cholesterol for intracellular delivery. Examples of drugs suitable for delivery by a CHEAP formulation include, but are not limited to, Rosiglitazone, Paclitaxel, and Tamoxifen. The efflux assay probe formulations described herein may alternatively comprise any of a number of other therapeutic agents such as, but not limited to, anticancer, antibacterial, antiviral, autoimmune, anti-inflammatory, and cardiovascular agents, antioxidants, or therapeutic peptides. In certain embodiments, the CHEAP particles incorporate about 0.6 mg of drug per 1 mg of particles. The drug-loaded CHEAP formulations release free drug molecules upon lysosomal degradation of the nanoparticles.

Other Methods of Use

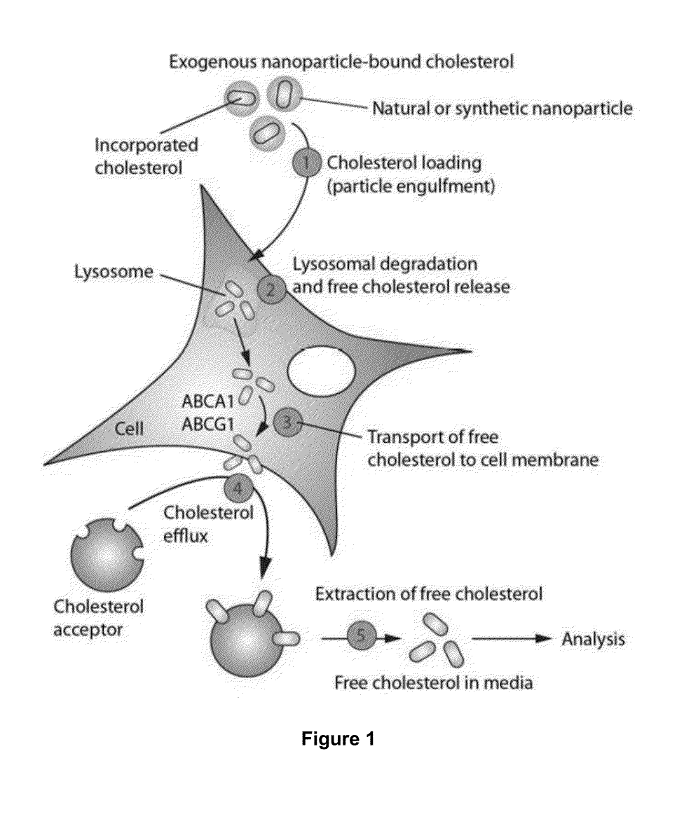

In another particular aspect, there are provided methods of using CHEAP formulations in the development of therapeutics in order to target biochemical pathways implicated in cholesterol trafficking. Such CHEAP formulations include labeled radioactively, fluorescently, or isotopically labeled cholesterol to monitor the pathways involved in cholesterol trafficking. In some embodiments, before cholesterol efflux can be monitored, reporter-labeled cholesterol (e.g., radioactively, fluorescently, or isotopically labeled) is loaded into the sample cells. Cholesterol loading is performed by incubating CHEAP nanoparticles having labeled cholesterol with the sample cells (Step 1). The nanoparticles are degraded by lysosomes, resulting in free cholesterol release (Step 2). Then, free cholesterol is transported to the cell membrane (Step 3), where it is removed from the cell membrane upon treatment with cholesterol acceptors (Step 4). This process is illustrated in FIG. 1. By exposing cholesterol-loaded cells to different cholesterol acceptors and environmental factors, the mechanisms and regulation of cholesterol efflux are elucidated. CHEAP formulations are thus useful for in-vitro cholesterol transport investigations.

In another aspect, the efflux assay probe formulations provided herein can be used to determine a lipid efflux profile by incorporating a variety of lipids into the formulation used for an assay. In some embodiments, assessing the lipid efflux profile of a subject includes measuring total cholesterol, cholesterol ester, HDL, LDL, intermediate density lipoprotein (IDL), very low density lipoprotein (VLDL), triglycerides, phospholipids selected from the group consisting of sphingolipids and phosphatidyl choline, and combinations thereof. In some embodiments, the sphingolipids are selected from the group consisting of sphingosines, ceramides, and sphingomyelins. Thus, in some embodiments, assessing the lipid efflux profile includes measuring cholesterol ester, sphingosines, ceramides, sphingomyelins, and phosphatidyl choline. In certain embodiments, assessing the lipid efflux profile involves analyzing only cholesterol. The lipid efflux profile can be obtained from macrophage cell types, macrophage-like cells, intact artery tissues, or in-vitro mobilization to plasma. Lipid efflux profiles can be used in methods to diagnose and/or identify subjects with a reverse cholesterol transport deficiency and distinguish responders from non-responders to treatments for an RCT deficiency-related condition.

In some embodiments, the formulations described herein can be used for prognosing, diagnosing, and/or predicting a response to a treatment of a condition associated with a deficiency in a reverse cholesterol transport pathway; for screening of compounds for treatment of a condition associated with RCT deficiency and/or assessing the risk of toxicity of a treatment of a condition associated with RCT deficiency; for identifying new druggable targets for the treatment of conditions associated with RCT deficiency; or to treat RCT-related diseases by, in part, administering to a subject in need thereof an effective amount of a therapeutic agent.

In one method, a condition associated with a deficiency in a RCT pathway can be diagnosed by: (i) providing a population of cells from the subject; (ii) loading the cells with an efflux assay probe formulation; (iii) assessing the lipid efflux profile; (iv) determining whether there is a deficiency in the RCT pathway of the subject, where the determining is based on the assessing of step (iii); and (v) if there is a deficiency determined from step (iv), diagnosing the subject as having a condition associated with a deficiency in a RCT pathway.

In another method, possible compounds for the treatment of a condition associated with reverse cholesterol transport deficiency can be screened by: (i) providing cells from a subject; (ii) contacting the cells with one or more compounds that are possible candidates for the treatment of a condition associated with reverse cholesterol transport deficiency; (iii) using an efflux assay probe formulation to assess the lipid efflux profile in the cells treated with the compound or a medium comprising the cells; and (iv) selecting the one or more compounds for treatment of the condition associated with reverse cholesterol transport deficiency, where the selecting is based on the assessing from step (iii). The compound may be a single compound or a combination of agents or compounds. The compound may also be a combination of agents or compounds together with some other intervention, such as a lifestyle change (e.g., change in diet or increase in exercise). The compound may already be approved for use in humans for the treatment or prevention of atherogenesis, arteriosclerosis, atherosclerosis, or other cholesterol-related diseases. The compound may be any compound, molecule, polymer, macromolecule, or molecular complex that can be screened for activity as described herein.

In another method, the risk of toxicity of a treatment of a condition associated with a reverse cholesterol transport deficiency can be assessed by: (i) providing cells from a subject; (ii) contacting the cells with one or more compounds that are used in the treatment of a condition associated with reverse cholesterol transport deficiency; (iii) using an efflux assay probe formulation to assess the lipid efflux profile in the cells treated with the compound or a medium comprising the cells; and (iv) determining the toxicity of a treatment of the condition associated with reverse cholesterol transport deficiency, where the determining is based on the assessing from step (iii). The compound may also be a combination of agents or compounds together with some other intervention, such as a lifestyle change (e.g., change in diet or increase in exercise). The compound may already be approved for use in humans for the treatment or prevention of atherogenesis, arteriosclerosis, atherosclerosis, or other cholesterol-related diseases. The compound may be any compound, molecule, polymer, macromolecule, or molecular complex that can be screened for activity as described herein.

In another method, a subject can be diagnosed with a condition associated with a deficiency in a reverse cholesterol transport pathway by: (i) administering to a subject an efflux assay probe formulation; (ii) assessing the lipid efflux profile in at least one cell from the subject; (iii) determining whether there is a deficiency in the reverse cholesterol transport pathway of the subject, where the determining is based on the assessing in step (ii); and (iv) diagnosing the subject as having a condition associated with a deficiency in a reverse cholesterol transport pathway, where the diagnosing is based on the determining in step (iii).

In another method, a subject can be diagnosed with a deficiency in the RCT pathway by: (i) isolating cells from a subject; (ii) contacting the cells with a compound that specifically modulates a reverse cholesterol transporter pathway; (iii) using an efflux assay probe formulation to assess the lipid efflux profile of the cells treated with the compound as compared to the lipid efflux profile of a control cell of the same type; and (iv) diagnosing the subject as having a condition associated with a deficiency in a reverse cholesterol transport pathway, where the diagnosing is based on the assessing in step (iii). In some embodiments, the modulator compound is a peptide.

As described, an efflux assay probe formulation can be utilized to assess the lipid efflux profile of a subject. The lipid efflux profile can be used in diagnosis or prognosis of a condition, patient selection for therapy, to monitor treatment, to modify therapeutic regimens, and/or to further optimize the selection of therapeutic agents which may be administered as one or a combination of agents. In some embodiments, the lipid efflux profile is compared to a predetermined threshold value, and the lipid efflux profile being above or below the predetermined threshold value is an indication that can be used in said diagnosis or prognosis. For example, a decrease of at least 20% or more of cholesterol ester and/or sphingolipids content in tissue can be used as an indication of a good prognosis, diagnosis, and/or treatment outcome. In some embodiments, the threshold is at least 20%, 25%, 30%, 40%, 50%, 60%, 70%, 80%, or 90% reduction of a lipid content in tissue when compared to a control sample. In some embodiments, the threshold is at least 20%, 25%, 30%, 40%, 50%, 60%, 70%, 80%, or 90% increase of a lipid content in tissue when compared to a control sample. In some embodiments, the threshold is at least 20%, 25%, 30%, 40%, 50%, 60%, 70%, 80%, or 90% reduction of a lipid content in plasma or serum when compared to a control sample. In some embodiments, the threshold is at least 20%, 25%, 30%, 40%, 50%, 60%, 70%, 80%, or 90% reduction of a lipid content in a cell when compared to a control sample. In some embodiments, the threshold is at least 20%, 25%, 30%, 40%, 50%, 60%, 70%, 80%, or 90% increase of a lipid content in a cell when compared to a control sample. In some embodiments, the lipid is cholesterol. In some embodiments, the lipid is a phospholipid. In some embodiments, the phospholipid is a sphingolipid. In some embodiments, the phospholipids are selected from the group consisting of sphingosines, ceramides, and sphingomyelins. Thus, in some embodiments, when the content of cholesterol ester, spingosines, ceramides, sphingomyelins, and phosphatidyl choline is above or below a predetermined threshold in tissue, blood, plasma, serum, and/or a cell, it is an indication that can be used in a diagnosis or prognosis of a condition, patient selection for therapy, to monitor treatment, to modify therapeutic regimens, and/or to further optimize the selection of therapeutic agents which may be administered as one or a combination of agents.

In some embodiments, the ratio of multiple lipid components in the lipid efflux profile can be used as an indication. For example, in some embodiments, the ratio of cholesterol ester to a sphingolipid can be used as an indication of a good prognosis, diagnosis, and/or treatment outcome. In certain embodiments, the ratio can be 0.001:1 to 1:1. In some embodiments, the ratio of one or more lipid components can be about 0.0001:1 to about 10:1, or about 0.001:1 to about 5:1, or about 0.01:1 to about 5:1, or about 0.1:1 to about 2:1, or about 0.2:1 to about 2:1, or about 0.5:1 to about 2:1, or about 0.1:1 to about 1:1. In some embodiments, the lipid components are cholesterol and a phospholipid. In some embodiments, the phospholipid is a sphingolipid. In some embodiments, the sphingolipids are selected from the group consisting of sphingosines, ceramids, and sphingomyelins.

Any of the methods described above may further comprise treating a RCT-related disease by administering to the subject in need thereof an effective amount of a therapeutic agent, such as a modulator that is specific for a reverse cholesterol transporter. The appropriate dosages for different subjects may be estimated using methods known by those of skill in the art. Effective dosages may be estimated initially from in-vitro assays. The therapeutic agents may be administered by any suitable route of administration known in the art, for example, by any of systemic, parenteral, inhalation spray, nebulized or aerosolized using aerosol propellants, nasal, vaginal, rectal, sublingual, urethral, by infusion, intraarterial, intrathecal, intrabronchial, subcutaneous, intradermal, intravenous, intracervical, intraabdominal, intracranial, intrapulmonary, intrathoracic, intratracheal, nasal routes, oral administration, drug delivery device, or by a dermal patch that delivers the therapeutic agent systemically, transdermally, or transbuccally. Therefore, the invention includes methods in which a subject is treated, diagnosed, and a treatment outcome is predicted. For example, a subject could be diagnosed with a condition, given a treatment, and after receiving the treatment obtain a prediction of the outcome of the treatment using the methods described herein. In this manner, the invention provides companion diagnostic and treatment methods.

Sampling

Some methods described herein involve analysis of one or more samples from an individual subject, the individual subject being any multi-cellular organism such as a mammal. In certain embodiments, the individual subject is a human.

The sample may be any suitable type that allows for the analysis intended. Samples may be obtained once or multiple times from a subject. Multiple samples may be obtained from different locations in the subject (e.g., blood samples, bone marrow samples, and/or atherosclerotic plaque samples), at different times from the subject (e.g., a series of samples taken to monitor response to treatment or to monitor for return of a pathological condition), or any combination thereof. These and other possible sampling protocols based on the sample time, location, and time of sampling allow for the detection and presence of pre-pathological or pathological cells, the measurement treatment response, and also the monitoring for disease.

When samples are obtained in series (e.g., a series of blood samples obtained after treatment), the samples may be obtained at fixed intervals, at intervals determined by the status of the most recent sample or samples, or by other characteristics of the subject, or some combination thereof. For example, samples may be obtained at intervals of approximately 1, 2, 3, or 4 weeks; at intervals of approximately 1, 2, 3, 4, 5, 6, 7, 8, 9, 10, or 11 months; at intervals of approximately 1, 2, 3, 4, 5, or more than 5 years; or some combination thereof. It will be appreciated that an interval may not be exact, according to a subject's availability for sampling and the availability of sampling facilities, thus approximate intervals corresponding to an intended interval scheme are encompassed by the invention. As an example, a subject who has undergone treatment for a cardiovascular disease may be sampled (e.g., by blood draw) relatively frequently (e.g., every month or every three months) for the first six months to a year after treatment, then, as treatment improves the condition, less frequently (e.g., at times between six months and a year) thereafter. If, however, any abnormalities or other circumstances are found in any of the intervening times, or during the sampling, sampling intervals may be modified.

Generally, the most easily obtained samples are fluid samples. Fluid samples include normal and pathologic bodily fluids and aspirates of those fluids. Fluid samples also comprise rinses of organs and cavities (lavage and perfusions). Bodily fluids include whole blood, bone marrow aspirate, synovial fluid, cerebrospinal fluid, saliva, sweat, tears, semen, sputum, mucus, menstrual blood, breast milk, urine, lymphatic fluid, amniotic fluid, placental fluid, and effusions such as cardiac effusion, joint effusion, pleural effusion, and peritoneal cavity effusion (ascites). Rinses can be obtained from numerous organs, body cavities, passage ways, ducts, and glands. Sites that can be rinsed include, but are not limited to, lungs (bronchial lavage), stomach (gastric lavage), gastrointestinal tract (gastrointestinal lavage), colon (colon lavage), vagina, bladder (bladder irrigation), breast duct (ductal lavage), oral, nasal, sinus cavities, and peritoneal cavity (peritoneal cavity perfusion).

Solid tissue samples may also be used, either alone or in conjunction with fluid samples. Solid samples may be derived from subjects by any method known in the art including, but not limited to, surgical specimens, biopsies, and tissue scrapings such as cheek scrapings. Surgical specimens include samples obtained during exploratory, cosmetic, reconstructive, or therapeutic surgery. Biopsy specimens can be obtained through numerous methods including, but not limited to, bite, brush, cone, core, cytological, aspiration, endoscopic, excisional, exploratory, fine needle aspiration, incisional, percutaneous, punch, stereotactic, and surface biopsy.

In certain embodiments, the sample is a bone marrow sample, a lymph node sample, a cerebrospinal fluid sample, or a blood sample. In some embodiments, combinations of bone marrow, lymph node, cerebrospinal fluid, and blood samples are used.

In one embodiment, a sample may be obtained from an apparently healthy subject during a routine checkup and analyzed so as to provide an assessment of the subject's general health status. In another embodiment, a sample may be taken to screen for commonly occurring diseases. Such screening may encompass testing for a single disease, a family of related diseases, or a general screening for multiple, unrelated diseases. Screening can be performed weekly, bi-weekly, monthly, bi-monthly, every several months, annually, or in several year intervals, and may replace or complement existing screening modalities.

In another embodiment, a subject with a known increased probability of disease occurrence may be monitored regularly to detect for the appearance of a particular disease or class of diseases. An increased probability of disease can be based on familial association, age, previous genetic testing results, or occupational, environmental, or therapeutic exposure to disease-causing agents. For example, the presence of inherent mutations that predispose subjects to a particular condition can be a factor to determine an increased probability of disease. Subjects with increased risk for specific diseases can be monitored regularly for the first signs of a condition. Monitoring can be performed weekly, bi-weekly, monthly, bi-monthly, every several months, yearly, or in several year intervals, or any combination thereof. Monitoring may replace or complement existing screening modalities. Through routine monitoring, early detection of the presence of disease may result in increased treatment options including treatments with lower toxicity and increased chance of disease control or cure.

Certain fluid samples can be analyzed in their native state with or without the addition of a diluent or buffer. Alternatively, fluid samples may be further processed to obtain enriched or purified discrete cell populations prior to analysis. Numerous enrichment and purification methodologies for bodily fluids are known in the art. A common method to separate cells from plasma in whole blood is through centrifugation using heparinized tubes. By incorporating a density gradient, further separation of the lymphocytes from the red blood cells can be achieved. A variety of density gradient media are known in the art including, but not limited to, sucrose, dextran, bovine serum albumin (BSA), FICOLL diatrizoate (Pharmacia), FICOLL metrizoate (Nycomed), PERCOLL (Pharmacia), metrizamide, and heavy salts such as cesium chloride. Alternatively, red blood cells can be removed through lysis with an agent such as ammonium chloride prior to centrifugation.

Whole blood can also be applied to filters that are engineered to contain pore sizes that select for the desired cell type or class. For example, rare pathogenic cells can be filtered out of diluted, whole blood following the lysis of red blood cells by using filters with pore sizes between 5 to 10 nm. Alternatively, whole blood can be separated into its constituent cells based on size, shape, deformability, or surface receptors or surface antigens by the use of a microfluidic device.

Select cell populations can also be enriched for or isolated from whole blood through positive or negative selection based on the binding of antibodies or other entities that recognize cell surface or cytoplasmic constituents.

Solid tissue samples may require the disruption of the extracellular matrix or tissue stroma and the release of single cells for analysis. Various techniques for this are known in the art including, but not limited to, enzymatic and mechanical degradation employed separately or in combination. Alternatively, single cells may be removed from solid tissue through microdissection such as laser capture microdissection.

The cells can be separated from body samples by centrifugation, elutriation, density gradient separation, apheresis, affinity selection, panning, FACS, centrifugation with Hypaque, solid supports (magnetic beads, beads in columns, or other surfaces) with attached antibodies, etc. By using antibodies specific for markers identified with particular cell types, a relatively homogeneous population of cells may be obtained. Alternatively, a heterogeneous cell population can be used. Cells can also be separated by using filters. Once a sample is obtained, it can be used directly, frozen, or maintained in appropriate culture medium for short periods of time.

In some embodiments, the cells are cultured post collection in media suitable for measuring RCT function, such as RPMI or DMEM, and in the presence or absence of serum such as fetal bovine serum, bovine serum, human serum, porcine serum, horse serum, or goat serum.

Kits

The efflux assay probe formulations described herein may be made available via a kit containing one or more key components. For instance, such a kit may comprise a first container containing a sphingomyelin and cholesterol mixture, a second container containing a biocompatible hydrophobic material, and, optionally, a sonicator. Many other kit variations are possible. A kit typically further includes instructions for using the components of the kit to practice the subject methods. The instructions for practicing the subject methods are generally recorded on a suitable recording medium. For example, the instructions may be present in the kits as a package insert or in the labeling of the container of the kit or components thereof. In other embodiments, the instructions are present as an electronic storage data file present on a suitable computer readable storage medium, such as a CD-ROM, flash drive, or diskette. In other embodiments, the actual instructions are not present in the kit, but means for obtaining the instructions from a remote source, such as via the internet, are provided. An example of this embodiment is a kit that includes a web address where the instructions can be viewed and/or from which the instructions can be downloaded. As with the instructions, the means for obtaining the instructions is recorded on a suitable substrate.

A kit may further comprise a software package for data analysis of the RCT pathway state, which may include reference profiles for comparison with a test profile. Additionally, the kits may include information, such as scientific literature references, package insert materials, clinical trial results, and/or summaries of these and the like, which indicate or establish the activities and/or advantages of the formulation and/or describe dosing, administration, side effects, drug interactions, or other information useful to a health care provider or other practitioner of the kits. Such information may be based on the results of various studies, such as studies using experimental animals involving in-vivo models and studies based on human clinical trials. By way of non-limiting example, kits described herein can be provided, marketed, and/or promoted to health care providers, including physicians, nurses, pharmacists, and the like. In some embodiments, kits may also be marketed directly to the consumer, for instance as kits for determining the user's own lipid efflux profile.

From the discussion and the Examples herein, one skilled in the art can ascertain the essential characteristics of this invention, and without departing from the spirit and scope thereof, can make various changes and modifications of the invention to adapt it to various usages and conditions. Various modifications of the invention in addition to those shown and described herein will be apparent to those skilled in the art from the foregoing description. Such modifications are also intended to fall within the scope of the appended claims.

### EXAMPLES

### Example 1

### CHEAP Formulation Optimization

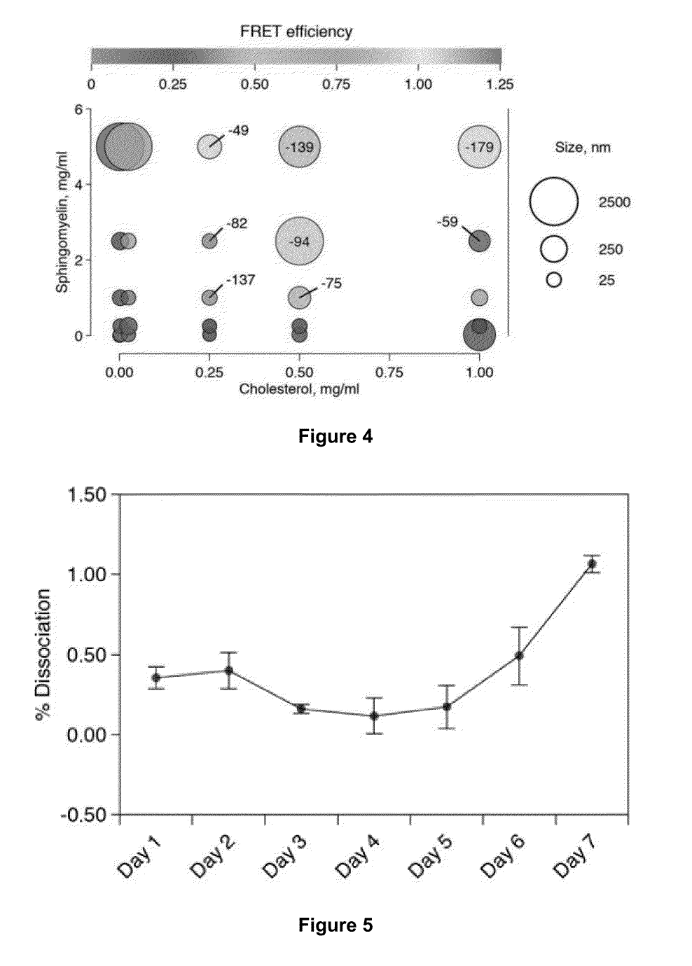

Twenty-five different CHEAP formulations which bear varying content of sphingomyelin/cholesterol were screened with respect to size, polydispersity index, zeta-potential and FRET efficiency. In order to enable FRET-capable CHEAP formulations, a fluorescently dyed polystyrene core was chosen to form a FRET pair with BODIPY-cholesterol, which was used for preparation of all formulations. The screening results are presented as a five-dimensional bubble chart in FIG. 4.

Formulations containing different sphingomyelin and cholesterol concentrations (plotted on the y- and x-axis, respectively) resulted in CHEAP formulations having particle sizes ranging from ˜2500 to 25 nm. Particles with size 25-50 nm were further tested. The results identified an optimized particle with mean size of ˜31 nm, FRET efficiency of ˜0.75, and zeta potential/polydispersity ratio of ˜−137. One CHEAP formulation produced with optimized parameters was comprised of 1.25 mg/mL sphingomyelin and 0.25 mg/mL cholesterol.

### Example 2

### In Vitro Testing

The CHEAP formulations were evaluated to determine their stability at different cell culture conditions. An optimized CHEAP formulation was synthesized as described above but with tritium (³H)-labeled cholesterol. No significant aggregation, changes in appearance, or cholesterol release was seen in the CHEAP formulation stored at room temperature or +4° C. This indicates CHEAP formulations have high shelf-life stability.

To determine the stability at the different cell culture conditions, the CHEAP formulations were subjected to dynamic dialysis in 50 kDa molecular weight cutoff dialysis tubing immersed in DMEM at 37° C. Cholesterol release as a consequence of particle dissociation was monitored by sampling of the dialysate followed by analysis of radioactivity on a scintillation counter. The results show that CHEAP was stable even after prolonged incubation (>7 days) at cell culture conditions. The amount of cholesterol released during the incubation period, shown by the amount of radioactivity found in the exterior DMEM, was less than 1%, as shown in FIG. 5.

### Example 3

### Drug Loading

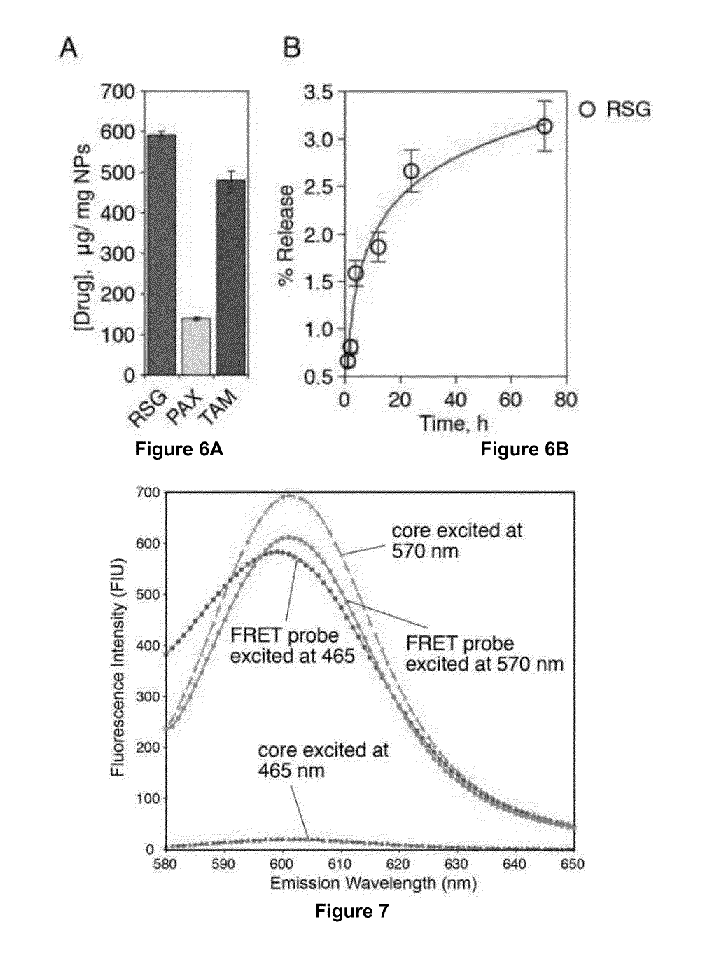

The incorporation into CHEAP of hydrophobic molecules other than cholesterol was studied. The intracellular delivery of hydrophobic drugs was conducted using a CHEAP formulation vehicle. Loading, as well as stability, of the drug-loaded CHEAP formulations was investigated on three different hydrophobic drugs. Rosiglitazone (RSG), Paclitaxel (PAX), and Tamoxifen (TAM) were used as model drugs for loading (FIG. 6A). The drugs showed high binding affinity towards the CHEAP formulation. In the present example, RSG was the most efficient, with CHEAP incorporating ˜0.6 mg of the drug per 1 mg of particles. The release experiments, monitored via LC-MS, showed very little RSG released even after 72 h at cell culture conditions (FIG. 6B).

### Example 4

### FRET Testing

The FRET capabilities of the CHEAP formulations allow for real-time cholesterol release monitoring in RCT assays. To confirm the occurrence of FRET, the fluorescence excitation/emission profiles of aqueous solutions of the CHEAP formulations composed of red fluorescence polystyrene nanoparticles and BODIPY-cholesterol were recorded. The fluorescence spectra of FIG. 7 show that, upon excitation at the wavelength of BODIPY-cholesterol (465 nm), the energy is transferred to the core of the CHEAP formulation nanoparticles, which stably emit at 605 nm. As shown in FIG. 7, the CHEAP formulations composed of a red fluorescent polystyrene core and sphingomyelin/BODIPY-cholesterol enable efficient FRET.

### Example 5

### Macrophage Staining

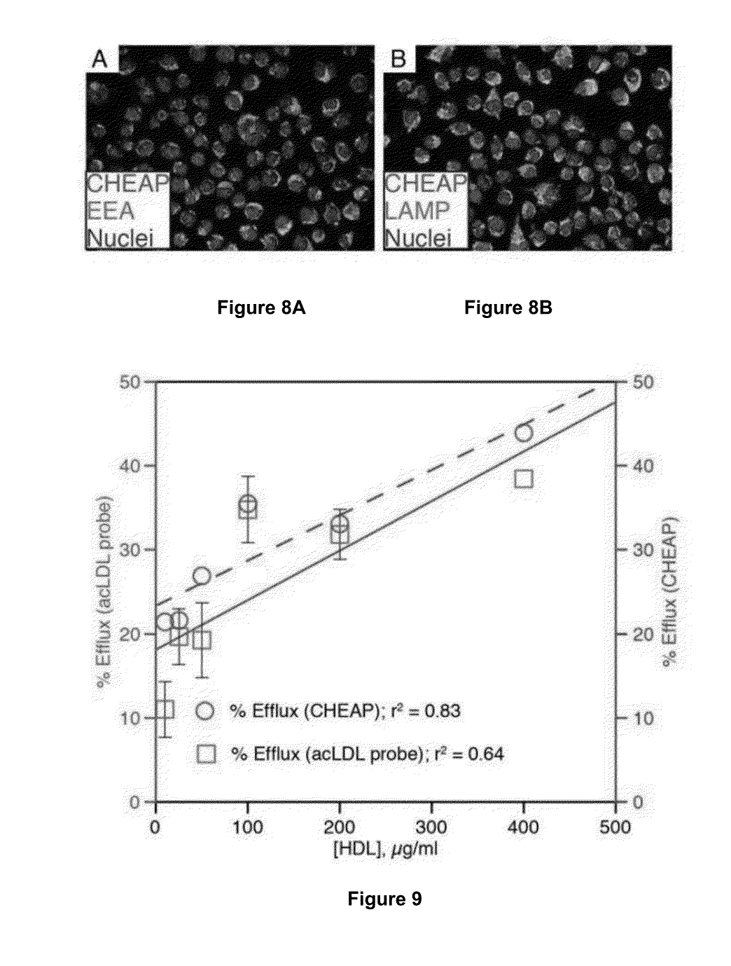

The polystyrene-core-based CHEAP nanoparticles are taken up by cells in a manner similar to natural LDL. To demonstrate that the CHEAP formulations accumulate in the lysosomal compartment of cells, the CHEAP formulations (red fluorescent core) were incubated with J774 cells for 4 h, followed by immunostaining against early endosomal antigen (EEA) or lysosomes (LAMP). The confocal microscope images of these cells show efficient co-staining of CHEAP, EEA, and lysosomes. FIGS. 8A-8B show macrophages treated with a fluorescent acLDL or a CHEAP formulation, and subsequently stained for endosomes (FIG. 8A) or for lysosomes (FIG. 8B). The CHEAP formulations appear red, nuclei appear blue, and the endosomes or lysosomes appear green.

### Example 6

### RCT Assay Comparison

The CHEAP formulations were assessed to confirm their efficient performance in RCT assays, as compared to acLDL probes. The CHEAP formulations and acLDL probes were loaded with radioactive ³H-cholesterol and compared in RCT assays, aided by treatment with different concentrations of human HDL, the cholesterol acceptor, for 4 h. The percent (%) cholesterol efflux, defined as the amount of cholesterol in the media divided by the total amount of cholesterol in the cells, was measured and charted as a function of HDL concentration, shown in FIG. 9. As seen from this figure, the CHEAP formulations behave in a desirable manner, quantitatively similar to acLDL probes.

While the invention has been described with reference to various embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof. Therefore, it is intended that the invention not be limited to a particular embodiment disclosed, but that the invention will include all embodiments falling within the scope of the claims.

What is claimed is:

1. A cholesterol efflux assay probe formulation, comprising at least one nanoparticle having a biocompatible hydrophobic core at least partially coated with a lipid-cholesterol mixture.

2. The formulation of claim 1, wherein the lipid-cholesterol mixture comprises a phospholipid and cholesterol.

3. The formulation of claim 2, wherein the phospholipid consists of sphingomyelin.

4. The formulation of claim 1, wherein the lipid-cholesterol mixture comprises cholesterol esters, triglycerides, or sphingolipids.

5. The formulation of claim 1, wherein the core comprises polystyrene.

6. The formulation of claim 1, wherein the at least one nanoparticle has a diameter in the range of about 25 nm to about 50 nm.

7. The formulation of claim 1, wherein the at least one nanoparticle has a diameter of about 31 nm.

8. The formulation of claim 1, wherein the core is at least partially porous to allow for passive adsorption of lipids and hydrophobic molecules on the surface of the formulation.

9. The formulation of claim 1, further including at least one of: a therapeutic agent, a diagnostic agent, or a contrast agent, at least partially encapsulated in the formulation.

10. The formulation of claim 1, wherein the formulation has a Zeta potential in the range of from about −10 mV to about −100 mV.

11. The formulation of claim 1, wherein the formulation is stable at cell culture conditions.

12. The formulation of claim 1, wherein the formulation comprises nanoparticles loaded with a drug selected from the group consisting of: cholesterol drugs, anticancer agents, antibacterial agents, antiviral agents, autoimmune agents, anti-inflammatory agents, cardiovascular agents, antioxidants, and therapeutic peptides.

13. The formulation of claim 11, wherein the drug is selected from the group consisting of Rosiglitazone, Paclitaxel, and Tamoxifen.

14. The formulation of claim 1, wherein the cholesterol is labeled with at least one of a radioactive label, a fluorescent label, or an isotopic label.

15. The formulation of claim 11, wherein the label is chosen from tritium or BODIPY.

16. The formulation of claim 1, wherein the core comprises red fluorescence polystyrene.

17. A method for making an efflux assay probe formulation, the method comprising:
i) forming a mixture of hydrophobic molecules;
ii) adding the mixture of step i) to an aqueous solution of 20-25 nm polystyrene latex nanoparticles to form a suspension; and
iii) sonicating the suspension of step ii) to yield an efflux assay probe formulation.

18. The method of claim 17, wherein the mixture of hydrophobic molecules is a lipid-cholesterol mixture.

19. The method of claim 18, wherein the lipid-cholesterol mixture comprises a phospholipid and cholesterol.

20. The method of claim 19, wherein the phospholipid consists of sphingomyelin.

21. The method of claim 18, wherein the lipid-cholesterol mixture further comprises cholesterol esters, triglycerides, or sphingolipids.

22. The method of claim 17, further comprising the step of labeling the cholesterol with at least one of a radioactive label, a fluorescent label, or an isotopical label.

23. The method of claim 22, wherein the cholesterol is labeled with tritium.

24. The method of claim 22, wherein the cholesterol is labeled with BODIPY.

25. The method of claim 17, further comprising the step of incorporating at least one of a therapeutic agent, a diagnostic agent, or a contrast agent into the formulation;
wherein the at least one of the therapeutic agent, diagnostic agent, or contrast agent is at least partially encapsulated in the formulation.

26. A method of conducting a reverse cholesterol transport assay, comprising:
loading cells with a formulation of claim 1, wherein the at least one nanoparticle is degraded by lysosomes to release cholesterol in the cells;
treating the cells with a cholesterol acceptor, wherein the cholesterol acceptor transports the cholesterol outside the cells to extracellular media; and
analyzing the cholesterol in the extracellular media.

27. The method of claim 26, wherein the cholesterol acceptor consists of HDL.

28. The method of claim 26, wherein the cholesterol is labeled with at least one of a radioactive label, a fluorescent label, or an isotopical label.

29. The method of claim 26, further comprising the step of conducting real-time monitoring of cholesterol efflux.

30. The method of claim 26, wherein the monitoring includes measuring Förster Resonance Energy Transfer effects.

31. A method of delivering a drug to a subject in need thereof, the method comprising:
incorporating an effective amount of a drug into a formulation of claim 1 to produce a drug-loaded nanoparticle composition; and
administering the drug-loaded nanoparticle composition to a subject in need thereof.

32. The method of claim 31, wherein the drug is selected from the group consisting of: cholesterol drugs, anticancer agents, antibacterial agents, antiviral agents, autoimmune agents, anti-inflammatory agents, cardiovascular agents, antioxidants, and therapeutic peptides.

33. The method of claim 31, wherein the drug is selected from the group consisting of Rosiglitazone, Paclitaxel, and Tamoxifen.

34. A method of delivering a therapeutic or diagnostic agent to a target site, the method comprising:
incorporating a therapeutic or diagnostic agent into a formulation of claim 1 to produce an agent-loaded formulation; and
delivering the agent-loaded formulation to the target site, wherein the delivery includes one or more of:
i) engulfment of the formulation within the target site;
ii) release of a diagnostic agent incorporated within the formulation to the target site; and
iii) release of a therapeutic agent incorporated within the formulation to the target site.

35. The method of claim 34, wherein the target site is atherosclerotic plaque tissue.

36. The method of claim 34, further including the step of imaging the target site.

37. A method of monitoring cholesterol efflux in real-time, the method comprising:
loading cells with nanoparticles comprising a fluorescent core and fluorescently labeled cholesterol; and
monitoring the FRET efficiency of the nanoparticles to monitor cholesterol efflux in real-time, wherein the FRET efficiency is inversely proportional to the distance between the cholesterol and the core.

38. The method of claim 37, wherein the core consists of red fluorescent polystyrene.

39. The method of claim 37, wherein the cholesterol is labeled with BODIPY.

40. The method of claim 37, wherein the nanoparticles further comprise sphingomyelin.

41. The method of claim 37, further comprising the step of incubating the cells with a cholesterol acceptor.

42. A reverse cholesterol transport assay, comprising:
a supply of cells;
a formulation of claim 1; and
one or more cholesterol acceptors.

43. The reserve cholesterol transport assay of claim 42, wherein the supply of cells is preloaded with the formulation.

44. The reverse cholesterol transport assay of claim 43, wherein the cholesterol is labeled with at least one of a fluorescent label, a radioactive label, or an isotopical label.

45. The reverse cholesterol transport assay of claim 43, wherein the one or more cholesterol acceptors comprises HDL.

46. The reverse cholesterol transport assay of claim 43, further comprising a fluorescence reader.

47. A method of assessing a lipid efflux profile, the method comprising:
incorporating cholesterol ester, HDL, LDL, IDL, VLDL, triglycerides, or combinations thereof, into a formulation of claim 1; and
conducting an efflux assay with the formulation to assess a lipid efflux profile.

48. A method of diagnosing a condition associated with a deficiency in a RCT pathway, comprising:
(i) providing a population of cells from the subject;
(ii) loading the cells with a formulation of claim 1;
(iii) assessing lipid efflux profile;
(iv) determining whether there is a deficiency in the RCT pathway of the subject, where the determining is based on the assessing of step (iii); and
(v) if there is a deficiency determined from step (iv), diagnosing the subject as having a condition associated with a deficiency in a RCT pathway.

49. A method of screening compounds for treatment of a condition associated with reverse cholesterol transport deficiency, comprising:
(i) providing cells from a subject;
(ii) contacting the cells with one or more compounds that are possible candidates for the treatment of a condition associated with reverse cholesterol transport deficiency;
(iii) using a formulation of claim 1 to assess the lipid efflux profile in the cells treated with the compound or a medium comprising the cells; and
(iv) selecting the one or more compounds for treatment of the condition associated with reverse cholesterol transport deficiency, where the selecting is based on the assessing from step (iii).

50. A method of assessing the risk of toxicity of a treatment of a condition associated with a reverse cholesterol transport deficiency, comprising:
(i) providing cells from a subject;
(ii) contacting the cells with one or more compounds that are used in the treatment of a condition associated with reverse cholesterol transport deficiency;
(iii) using a formulation of claim 1 to assess the lipid efflux profile in the cells treated with the compound or a medium comprising the cells; and
(iv) determining the toxicity of a treatment of the condition associated with reverse cholesterol transport deficiency, where the determining is based on the assessing from step (iii).

51. A method of diagnosing a condition associated with a deficiency in a reverse cholesterol transport pathway in a subject, comprising:
(i) administering to a subject a formulation of claim 1;
(ii) assessing the lipid efflux profile in at least one cell from the subject;
(iii) determining whether there is a deficiency in the reverse cholesterol transport pathway of the subject, where the determining is based on the assessing in step (ii); and
(iv) diagnosing the subject as having a condition associated with a deficiency in a reverse cholesterol transport pathway, where the diagnosing is based on the determining in step (iii).

52. A method of diagnosing a subject with a deficiency in the RCT pathway, comprising:
(i) isolating cells from a subject;
(ii) contacting the cells with a compound that specifically modulates a reverse cholesterol transporter pathway;
(iii) using a formulation of claim 1 to assess the lipid efflux profile of the cells treated with the compound as compared to the lipid efflux profile of a control cell of the same type; and
(iv) diagnosing the subject as having a condition associated with a deficiency in a reverse cholesterol transport pathway, where the diagnosing is based on the assessing in step (iii).

53. A kit for the preparation of a CHEAP formulation comprising:
a first container containing a mixture of sphingomyelin and cholesterol;
a second container containing a biocompatible hydrophobic material; and
optionally, a sonicator.

54. The kit of claim 53, wherein the biocompatible hydrophobic material consists of polystyrene.

55. The kit of claim 53, further comprising one or more of: a therapeutic agent, a diagnostic agent, or a diagnostic agent.
